(19) United States
(12) Patent Application Publication
(10) Pub. No.: US 2009/0216282 A1
(43) Pub. Date: Aug. 27, 2009

(54) SYSTEMS AND METHODS FOR RETAINING A PLATE TO A SUBSTRATE WITH AN ASYNCHRONOUS THREAD FORM

(76) Inventors: Doris M. Blake, Delray Beach, FL (US); Trace Cawley, Boca Raton, FL (US)

Correspondence Address: Clements Bernard PLLC, 1901 Roxborough Road, Suite 300, Charlotte, NC, 28211, US

(21) Appl. No.: 12/395,766

(22) Filed: Mar. 2, 2009

Related U.S. Application Data
(63) Continuation-in-part of application No. 11/804,545, filed on May 18, 2007.
(60) Provisional application No. 61/033,077, filed on Mar. 3, 2008.

(51) Int. Cl. A61B 17/80; A61B 17/86

(52) U.S. Cl. 606/286; 606/305

(57) ABSTRACT

The present disclosure provides systems and methods for retaining a plate, such as a cervical plate, to a substrate, such as bone, through a locking screw with upper asynchronous threads and lower cancellous threads. The present invention utilizes the locking screw with cancellous-type threads to draw the screw into the substrate. The screw is operable to retain a plate to the substrate and is configured to gall and lock to the plate through the upper threads which are out-of-phase with corresponding threads on the plate thereby preventing reverse threading or backing out of the screw.

### CROSS-REFERENCE TO RELATED APPLICATION(S)

The present non-provisional patent application is a continuation-in-part of commonly assigned U.S. patent application Ser. No. 11/804,545, filed May 18, 2007, and entitled “CERVICAL PLATE LOCKING MECHANISM AND ASSOCIATED SURGICAL METHOD,” the contents of which are herein incorporated by reference. Additionally, the present non-provisional patent application claims priority to U.S. Provisional Patent Application Ser. No. 61/033,077, filed Mar. 3, 2008, and entitled “SYSTEMS AND METHODS FOR RETAINING A PLATE TO A SUBSTRATE WITH AN ASYNCHRONOUS THREAD FORM,” the contents of which are herein incorporated by reference.

### FIELD OF THE INVENTION

The present invention relates generally to surgically implanted devices and associated surgical implantation methods. More particularly, the present invention provides systems and methods for retaining a plate, such as a cervical plate, to a substrate, such as bone, through a screw with asynchronous upper threads that engage and lock to the plate and lower cancellous threads that engage the substrate.

### BACKGROUND OF THE INVENTION

The vertebrae of the human spine are generally arranged in a column, with an intervertebral disc disposed between each. These intervertebral discs transmit forces and perform a “cushioning” function. As a result of the stresses and strains continuously applied to the intervertebral discs, as well as disease, degeneration and/or deformity is relatively common. Typically, diseased, degenerated, and/or deformed intervertebral discs are treated by removal and the insertion of an implant, anatomical (i.e. a bone graft) or mechanical (i.e. a biocompatible insert), in the associated intervertebral space. The adjacent vertebrae are preferably immobilized using a plate, such as a cervical plate, during bone graft or biocompatible insert placement and subsequently until they fuse, for example. The cervical plate is an exemplary application of a surgical technique whereby a plate is coupled to a substrate. Other types of plates can also be used in a variety of surgical applications such as dental implants and the like.

Conventional cervical plates typically include a plurality of screw holes and one or more access holes, through which one or more bone grafts or other biocompatible inserts are placed. These cervical plates can span one or multiple levels, with a level defined by the presence of an intervertebral space, and are secured to the vertebrae of the spine using a plurality of bone screws. The cervical plate is used to aid in the fusion of adjacent vertebrae, providing required stability after damaged intervertebral disc has been removed from and new bone graft has been placed into the intervertebral space(s) of interest.

One problem that is often encountered is that these cervical plate screws tend to “back out” over time absent some sort of locking mechanism. This reverse threading or backing out is obviously problematic. Various cervical plate screw locking mechanisms have been designed and manufactured to alleviate this “backing out” problem; however, such cervical plate screw locking mechanisms have typically been ineffective and/or overly complicated, sacrificing the desirable low profile nature of the cervical plate, for example. Thus, an improved cervical plate and cervical plate screw locking mechanism are still needed in the art.

### BRIEF SUMMARY OF THE INVENTION

In various exemplary embodiments, the present invention provides systems and methods for retaining a plate, such as a cervical plate, to a substrate, such as bone, through a locking screw with upper asynchronous threads and lower cancellous threads. The present invention utilizes the locking screw with cancellous-type threads to draw the screw into the substrate. The screw is operable to retain a plate to the substrate and is configured to gall and lock to the plate through the upper threads which are out-of-phase with corresponding threads on the plate thereby preventing reverse threading or backing out of the screw.

In an exemplary embodiment of the present invention, a system for retaining a plate to a substrate with an asynchronous thread form includes a plate defining one or more holes each configured to receive a locking screw, wherein the plate includes an outer surface, an interior portion, and an inner surface, each of the one or more holes having an inside diameter that is smaller adjacent to the outer surface than at the interior portion and partial threads disposed in the inner surface; and one or more locking screws, each including a first threaded portion configured to pass through one of the one or more holes of the plate and securely engage a substrate; and a second threaded portion configured to pass through one of the one or more holes of the plate and securely engage the partial threads, wherein the first threaded portion is asynchronous from the second threaded portion. The one or more locking screws each further include a head portion attached to the second threaded portion configured to securely engage a lip structure associated with one of the one or more holes of the plate. The second threaded portion includes threads out-of-phase with the partial threads disposed in the inner surface. The second threaded portion is configured to gall and lock causing partial buckling with the partial threads responsive to an applied torque thereby preventing reverse threading or backing out of the locking screw from the plate. The second threaded portion includes threads with one or more different values for pitch, major diameter, minor diameter, root to crest distance, and thread angle from the partial threads disposed in the inner surface. The second threaded portion also includes threads with one or more different values for pitch, major diameter, minor diameter, root to crest distance, and thread angle from the first threaded portion. The first threaded portion includes a cancellous type thread operable to draw the screw into the substrate. Optionally, the substrate includes a bone, and wherein the plate includes a cervical plate. Alternatively, each of the one or more holes defined by the plate is configured to receive one of the one or more locking screws one of perpendicularly and at an angle.

In another exemplary embodiment of the present invention, a locking screw operable to retain a plate to a substrate includes an elongated member including an end portion operable to engage a substrate, a middle portion, and an upper portion; first threads on the middle portion of the elongated member; second threads on the upper portion of the elongated member, wherein the first threads and the second threads are asynchronous; and a head disposed to the elongated member. The head includes a notch configured to securely engage a lip structure associated with a hole in the plate. The second threads are out-of-phase with partial threads disposed in an inner surface of a hole in the plate. The second threads are configured to gall and lock causing partial buckling with the partial threads responsive to an applied torque thereby preventing reverse threading or backing out of the locking screw from the plate. The second threads include one or more different values for pitch, major diameter, minor diameter, root to crest distance, and thread angle from partial threads disposed in an inner surface of a hole in the plate. The second threads include one or more different values for pitch, major diameter, minor diameter, root to crest distance, and thread angle from the first threads. Optionally, the first threads include cancellous type threads operable to draw the screw into the substrate, wherein the substrate includes a bone, and wherein the plate includes a cervical plate.

In yet another exemplary embodiment of the present invention, a method for retaining a plate to a substrate with an asynchronous thread form includes positioning plate defining one or more holes on a substrate, wherein the one or more holes each configured to receive a locking screw, wherein the plate included an outer surface, an interior portion, and an inner surface, each of the one or more holes having an inside diameter that is smaller adjacent to the outer surface than at the interior portion and partial threads disposed in the inner surface; positioning a locking screw within one of the one or more holes in the plate; applying a torque to a head portion of the locking screw to draw the locking screw into the substrate through a first threaded portion of the locking screw; and galling and locking a second threaded portion of the locking screw to a partial thread in the one of the one or more holes in the plate as the locking screw is drawn into the substrate. The method further includes implementing a partial buckling between the second threaded portion of the locking screw and the partial threads of the plate based on asynchronous threading of the second threaded portion in relation to the partial threads. The second threaded portion includes threads with one or more different values for pitch, major diameter, minor diameter, root to crest distance, and thread angle from the partial threads. The second threaded portion includes threads with one or more different values for pitch, major diameter, minor diameter, root to crest distance, and thread angle from the first threaded portion.

### BRIEF DESCRIPTION OF THE DRAWINGS

The present invention is illustrated and described herein with reference to the various drawings, in which like reference numbers denote like method steps and/or system components, respectively, and in which:

FIG. 1 is an exploded perspective view of one exemplary embodiment of the cervical plate locking mechanism of the present invention (being installed using a keyed screwdriver or the like), the cervical plate locking mechanism including both novel plate and novel locking screw designs;

FIG. 2 is an exploded perspective view of one exemplary embodiment of the novel locking screw design of FIG. 1, the locking screw including a head portion that incorporates a plurality of petal structures that are outwardly biased by an internally-disposed c-ring or the like;

FIG. 3 is a perspective view of the novel locking screw design of FIGS. 1 and 2, the locking screw being in its “as inserted” state, with the c-ring being installed and the head portion being compressed;

FIG. 4 is a partial cross-sectional view of the cervical plate locking mechanism of FIG. 1, the novel locking screw of FIGS. 1-3 in the process of being inserted into the novel plate of FIG. 1;

FIG. 5 is a partial cross-sectional view of the cervical plate locking mechanism of FIGS. 1 and 4, the novel locking screw of FIGS. 1-4 being fully inserted into the novel plate of FIGS. 1 and 4;

FIG. 6 is a partial cross-sectional view of the cervical plate locking mechanism of FIGS. 1, 4, and 5, the novel locking screws of FIGS. 1-5 being inserted into the novel plate of FIGS. 1, 4, and 5 at various exemplary angles;

FIG. 7 is a perspective view of a locking screw for fixedly securing a plate to a substrate with a head, upper threads, and lower cancellous threads according to an exemplary embodiment of the present invention;

FIG. 8 is a partial cross-sectional view of a cervical plate with an opening operable to receive the locking screw of FIG. 7 according to an exemplary embodiment of the present invention;

FIG. 9 is a partial cross-sectional view of a locking screw/cervical plate configuration with the screw placed horizontally through the opening of the plate according to an exemplary embodiment of the present invention;

FIG. 10 is a partial cross-sectional view of a locking screw/cervical plate with a poly-axial placement of the screw through the opening of the plate according to an exemplary embodiment of the present invention;

FIG. 11 is a flowchart of a plate retaining mechanism for retaining a plate to a substrate with an asynchronous thread form according to an exemplary embodiment of the present invention; and

FIG. 12 is an exploded perspective view of a cervical plate with a plurality of recessed screw holes disposed there through for receiving a locking screw of FIGS. 7-11 according to an exemplary embodiment of the present invention.

### DETAILED DESCRIPTION OF THE INVENTION

In various exemplary embodiments, the present invention provides systems and methods for retaining a plate, such as a cervical plate, to a substrate, such as bone, through a locking screw with upper asynchronous threads and lower cancellous threads. The present invention utilizes the locking screw with cancellous-type threads to draw the screw into the substrate. The screw is operable to retain a plate to the substrate and is configured to gall and lock to the plate through the upper threads which are out-of-phase with corresponding threads on the plate thereby preventing reverse threading or backing out of the screw.

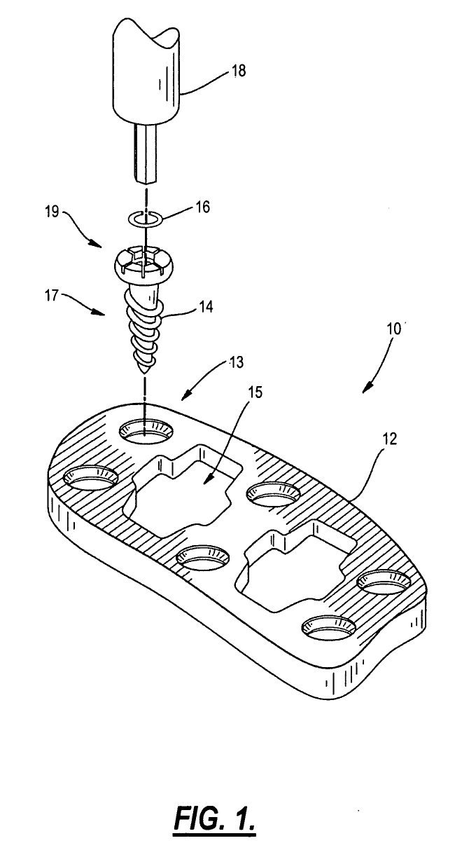

FIG. 1 is an exploded perspective view of one exemplary embodiment of the cervical plate locking mechanism 10 of the present invention (being installed using a keyed screwdriver 18 or the like), the cervical plate locking mechanism 10 including both novel plate and novel locking screw designs, as are described in greater detail herein below. Specifically, the cervical plate locking mechanism 10 includes a plate 12 that is configured to be securely fixed to adjacent vertebrae of the cervical spine or the like via one or more locking screws 14 and one or more c-rings 16. The keyed screwdriver 18 is used to drive the one or more locking screws 14 through the plate 12 and into the adjacent vertebrae.

The plate 12 includes one or more screw-receiving holes 13 and, optionally, one or more access holes 15 for the placement of one or more bone grafts, biocompatible inserts, or the like. Preferably, the plate 12 is manufactured from a biocompatible material and is sized such that it achieves its intended purpose. Material, shape, and size selection is well known to those of ordinary skill in the art. Each of the one or more locking screws 14 includes a threaded portion 17 and a head portion 19. The threaded portion 17 of each of the one or more locking screws 14 is configured to pass through the one or more screw-receiving holes 13 of the plate 12 and securely fix the plate 12 to the adjacent vertebrae.

The head portion 19 of each of the one or more locking screws 14 is configured to securely engage each of the one or more locking screws 14 with the plate 12. As described in greater detail herein below, the head portion 19 of each of the one or more locking screws 14 is outwardly biased by the c-ring 16, or by another comparable mechanism, which is selectively compressed, inserted into the head portion 19 of a given locking screw 14, and then allowed to expand. The c-ring 16, or other comparable mechanism, and the head portion 19 of the given locking screw 14 are again compressed and subsequently allowed to expand as they are inserted into a given screw-receiving hole 13 of the plate 12. Specifically, the head portion 19 of the given locking screw 14 is allowed to expand in the receiving well of the given screw-receiving hole 13. This insertion is accomplished using a matching flat, triangle, square, star, hexagon, octagon, or other keyed screwdriver 18, as appropriate. Preferably, the shape of the outside of the head portion 19 of each of the locking screws 14 substantially corresponds to the shape of the inside of the associated receiving well, although this is not a requirement.

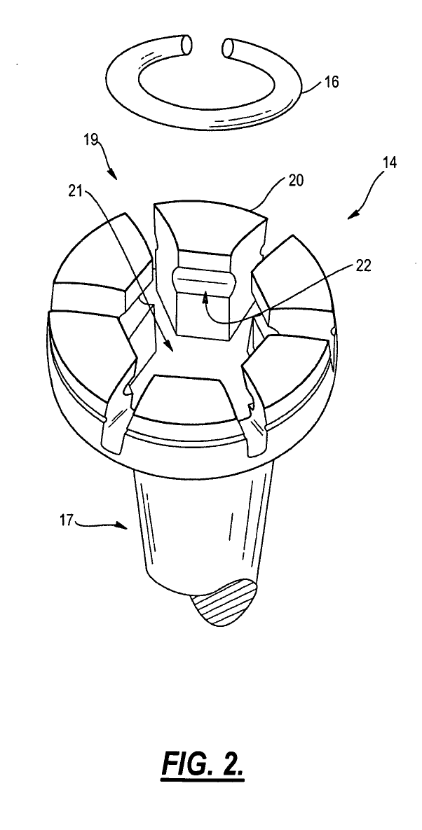

FIG. 2 is an exploded perspective view of one exemplary embodiment of the novel locking screw design of FIG. 1, the locking screw 14 including a head portion 19 that incorporates a plurality of petal structures 20 that are outwardly biased by the internally-disposed c-ring 16 or the like. As described above, the c-ring 16, or other comparable mechanism, is selectively compressed, inserted into the head portion 19 of a given locking screw 14, and then allowed to expand. The c-ring 16, or other comparable mechanism, and the head portion 19 of the given locking screw 14 are again compressed and subsequently allowed to expand as they are inserted into a given screw-receiving hole 13 (FIG. 1) of the plate 12 (FIG. 1).

Specifically, the head portion 19 of the given locking screw 14 is allowed to expand in the receiving well of the given screw-receiving hole 13. This insertion is accomplished using a matching flat, triangle, square, star, hexagon, octagon, or other keyed screwdriver 18 (FIG. 1), as appropriate. Preferably, the shape of the outside of the head portion 19 of each of the locking screws 14 substantially corresponds to the shape of the inside of the associated receiving well, although this is not a requirement. Accordingly, the head portion 19 of each of the locking screws 14 includes a plurality of concentrically-arranged petal structures 20 that are disposed around a central driver bore 21 that has a shape corresponding to that of the keyed screwdriver 18.

In one exemplary embodiment, the plurality of petal structures 20 are formed by cutting concentrically-arranged slots into the head portion 19 of the locking screw 14. Thus, the plurality of petal structures 20 are integrally formed with the head portion 19 of the locking screw 14. Alternatively, the plurality of petal structures 20 are formed separately and then joined to the head portion 19 of the locking screw 14. The material characteristics or configuration of the plurality of petal structures 20 may impart the plurality of petal structures 20 with an inherent outward bias, independent of the c-ring 16 or other comparable mechanism, although this is not required. Preferably, the plurality of petal structures 20 define an inner groove 22 that is configured to receive and retain the c-ring 16 or other comparable mechanism within the head portion 19 of the locking screw 14.

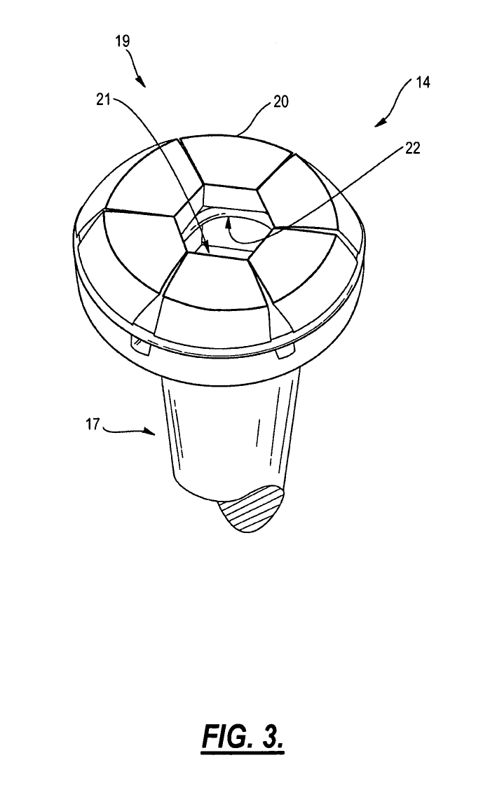

FIG. 2 illustrates the head portion 19 of the locking screw 14 in an “unlocked” configuration, with the plurality of petal structures 20 being “open,” either due to the eventual insertion of the c-ring 16 or other comparable mechanism, or inherently. FIG. 3 illustrates the head portion 19 of the locking screw 14 in a “locked” configuration, with the plurality of petal structures 20 being “closed,” either inherently or due to the eventual insertion of the head portion 19 of the locking screw 14 into a receiving well.

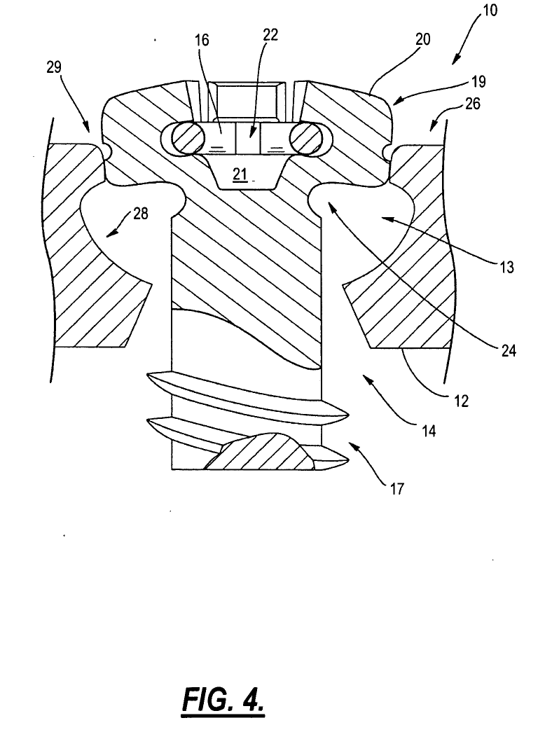

FIG. 4 is a partial cross-sectional view of the cervical plate locking mechanism 10 of FIG. 1, the novel locking screw 14 of FIGS. 1-3 in the process of being inserted into the novel plate 12 of FIG. 1. It should be noted that the head portion 19 of the locking screw 14, and specifically the lower, outer portion of each of the plurality of petal structures 20, optionally incorporates a recessed or otherwise weakened area 24, or flexure, in order to facilitate the flexibility and/or outward biasing of the plurality of petal structures 20 by the c-ring 16 or other comparable mechanism, after it is inserted into the inner groove 22 that is manufactured into the middle, inner portion of each of the plurality of petal structures 20.

Each of the one or more screw-receiving holes 13 of the plate 12 includes an annular lip structure 26 through which the head portions 19 of the locking screws 14 are inserted (with a compression-expansion action). This annular lip structure 26 serves to retain the head portion 19 of the given locking screw 14 once it is fully inserted and expanded, thereby preventing the reverse threading or backing out of the locking screw 14. Optionally, the inner annular surface 28 of each of the screw-receiving holes 13 of the plate 12 is curved in a generally concave manner, but shaped such that the lead-in torque of a given locking screw 14 is less than the lead-out torque or the locking screw 14, i.e. the inner annular surface angles adjacent to the outer surface 29 of the plate 12 (at the “top” and “bottom” of the lip structure 26) vary as experienced by an inserted locking screw 14 versus a removed locking screw 14, with the “top” angle being greater (more vertical or steep) and the “bottom” angle being smaller (more horizontal or shallow), for example.

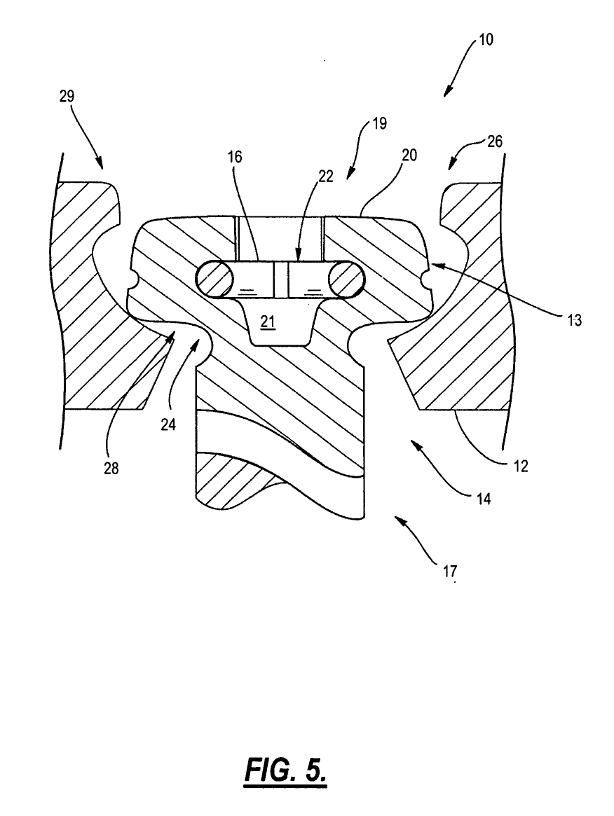

FIG. 5 is a partial cross-sectional view of the cervical plate locking mechanism 10 of FIGS. 1 and 4, the novel locking screw 14 of FIGS. 1-4 being fully inserted into the novel plate 12 of FIGS. 1 and 4. Again, it should be noted that the head portion 19 of the locking screw 14, and specifically the lower, outer portion of each of the plurality of petal structures 20, optionally incorporates a recessed or otherwise weakened area 24, or flexure, in order to facilitate the flexibility and/or outward biasing of the plurality of petal structures 20 by the c-ring 16 or other comparable mechanism, after it is inserted into the inner groove 22 that is manufactured into the middle, inner portion of each of the plurality of petal structures 20. Each of the one or more screw-receiving holes 13 of the plate 12 includes an annular lip structure 26 through which the head portions 19 of the locking screws 14 are inserted (with a compression-expansion action).

This annular lip structure 26 serves to retain the head portion 19 of the given locking screw 14 once it is fully inserted and expanded, as illustrated, thereby preventing the reverse threading or backing out of the locking screw 14. Optionally, the inner annular surface 28 of each of the screw-receiving holes 13 of the plate 12 is curved in a generally concave manner, but shaped such that the lead-in torque of a given locking screw 14 is less than the lead-out torque or the locking screw 14, i.e. the inner annular surface angles adjacent to the outer surface 29 of the plate 12 (at the “top” and “bottom” of the lip structure 26) vary as experienced by an inserted locking screw 14 versus a removed locking screw 14, with the “top” angle being greater (more vertical or steep) and the “bottom” angle being smaller (more horizontal or shallow), for example.

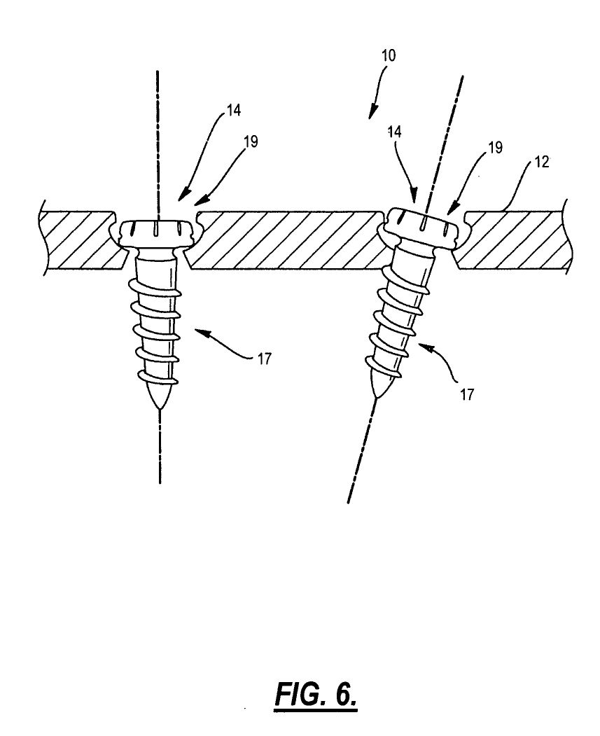

FIG. 6 is a partial cross-sectional view of the cervical plate locking mechanism 10 of FIGS. 1, 4, and 5, the novel locking screws 14 of FIGS. 1-5 being inserted into the novel plate 12 of FIGS. 1, 4, and 5 at various exemplary angles relative to both the plate 12 and the underlying vertebrae. In this embodiment, each of the receiving wells may be asymmetrical in shape such that the head portion 19 of each of the locking screws 14 snugly and securely engages the receiving well, although this is not necessarily illustrated. In other words, each of the receiving wells may be appropriately angled in the plate 12 in order to receive each of the angled locking screws 14.

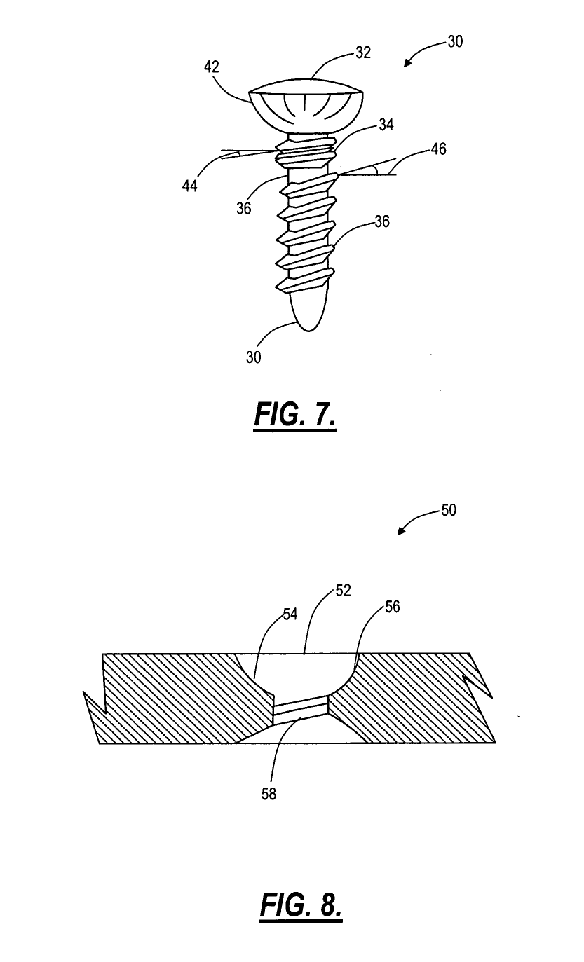

FIG. 7 is a perspective view of a locking screw 30 for fixedly securing a plate to a substrate with a head 32, upper thread 34, and lower cancellous threads 36 according to an exemplary embodiment of the present invention. Each of the threads 34, 36 is on an elongated member 38. The elongated member 38 includes a pointed end 40 at one end and the head 32 at the opposite end. The pointed end 40 can include a sharp point or the like to engage the substrate. The screw 30 is configured to fixedly secure a plate to a substrate. For example, the screw 30 can be utilized with a cervical plate to fixedly secure the plate to bone in the spinal cord. The pointed end 40 is operable to engage the substrate for implantation responsive to torque applied to the head 32.

The head 32 includes an underbody 42 that is shaped to fit within an annular opening in the plate as the screw 30 is embedded in the substrate. Correspondingly, the head 32 and the underbody 42 include an annular shape. Other shapes are also contemplated. The head 32 can be engaged with a matching flat, triangle, square, star, hexagon, octagon, or other keyed screwdriver, as appropriate, to apply torque to the screw 30 for implantation in the substrate.

The cancellous threads 36 are configured to draw the screw into the substrate while drawing the upper threads 34 to the plate responsive to applied torque to the head 32. The cancellous threads 36 are generally designed for placement in cancellous bone. The pullout strength of the screw 30 is proportional to the amount of metal-bone contact. Because cancellous bone is porous, the cancellous threads 36 have to be longer than for cortical screws to achieve the same degree of metal-bone contact and thus have the same pullout strength as cortical screws.

The upper threads 34 are designed asynchronously from the cancellous threads 36. Asynchronously means the upper thread 34 and the cancellous thread 34 have a differing pitch, major diameter, minor diameter, root to crest distance, and/or thread angle. The pitch is the distance, parallel to the screw axis, between corresponding points on adjacent thread forms having uniform spacing. The major diameter is the largest (outside) diameter of a screw thread. The minor diameter is the smallest diameter of a screw thread. The root to crest distance is the distance from the outside of a thread to the inside of the same thread. The thread angle is the angle of the threads relative to the elongated rod 38.

The upper threads 34 include a thread angle 44 and the cancellous threads 36 include a thread angle 46. Here, the thread angle 44 is less than the thread angle 46. As described above, the cancellous threads 36 have a higher thread angle 46 to enable more surface area on the threads 36 thereby providing more metal-bone contact. The upper threads 34 require less of an angle for the thread angle 44 to engage corresponding threads on the plate. As described herein, the upper threads 34 and the cancellous threads 36 are in an asynchronous relationship whereby the thread angles 44, 46 differ.

The upper threads 34 also have a slight out-of-phase relationship with the corresponding threads on the plate, i.e. the upper threads 34 are asynchronous from the corresponding threads on the plate. The cancellous threads 36 draw the elongated member 38 into the substrate. As the elongated member 38 is drawn into the substrate, the upper threads 34 are drawn into the partial corresponding threads in the plate. Accordingly, the upper threads 34 gall and lock into the plate because the upper thread 34 pitch is out-of-phase to a pitch on threads on the plate, for example. The upper threads 34 implement a partial buckling with the corresponding threads on the plate. The buckling and gall and lock action enable an effective lock between the screw 30 and the plate thereby preventing reverse threading or backing out of the screw.

FIG. 8 is a partial cross-sectional view of a cervical plate 50 with an annular opening operable 52 to receive the locking screw 30 of FIG. 7 according to an exemplary embodiment of the present invention. For example, the plate 50 can be a cervical plate such as the plate 12 in FIG. 1 used for support of the spine in spinal surgery. The present invention also contemplates other types of plates or implants connected to a bony structure through the locking screw 30 as are known in the art.

The screw 30 is placed in the opening 52 and the cancellous threads 36 extend through the opening 52 to an adjacent substrate, e.g. bone. The opening 50 includes side walls 54, 56 shaped to mate with the underbody 42 of the screw head 32. This annular opening 52 serves to retain the head 32 of the given locking screw 30 once it is fully inserted and expanded, thereby preventing the reverse threading or backing out of the locking screw 30 and preventing the plate 50 from releasing from the substrate. Optionally, the side walls 54, 56 of each of the openings 52 of the plate 50 are curved in a generally concave manner, but shaped such that the lead-in torque of a given locking screw 30 is less than the lead-out torque or the locking screw 30, i.e. the inner annular surface angles adjacent to the side walls 54, 56 of the plate 50 (at the “top” and “bottom” of the opening 52) vary as experienced by an inserted locking screw 50 versus a removed locking screw 50, with the “top” angle being greater (more vertical or steep) and the “bottom” angle being smaller (more horizontal or shallow), for example.

The plate 50 includes partial threads 58 in the plate opening 52. As described herein, the partial threads 58 are configured with a pitch that is out-of-phase from the upper threads 54 of the screw 30. Additionally, the major diameter, minor diameter, root to crest distance, and/or thread angle of the partial threads 58 can also differ from the corresponding upper threads 34. The partial threads 58 are configured to lock the screw 30 to the plate 50 through the upper threads 34. Additionally, the head 32 prevents the plate 50 from disengaging the screw 30 when implanted in the substrate.

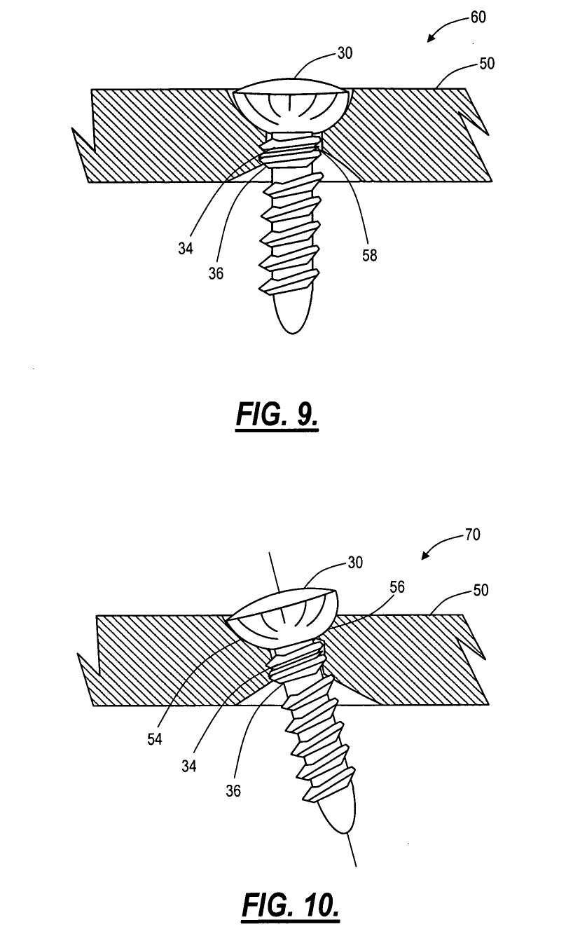

FIG. 9 is a partial cross-sectional view of a screw/plate configuration 60 with the screw 30 placed horizontally through the opening 52 of the plate 50 according to an exemplary embodiment of the present invention. The screw/plate configuration 60 represents a mono-axial placement of the screw 30. Here, the upper thread 34 and the upper threads 36 of the screw 30 are engaged to the partial threads 58 of the plate 50 causing an effective lock between the screw 30 and the plate 50. Optionally, the head 32 is substantially flush with the plate 50 when fully embedded in the substrate.

FIG. 10 is a partial cross-sectional view of a screw/plate configuration 70 with a poly-axial placement of the screw 30 through the opening 52 of the plate 50 according to an exemplary embodiment of the present invention. The side walls 54, 56 of the plate 50 can be shaped to mate with the underbody 42 of the screw in a polyaxial configuration as is typically used in spinal configurations. The polyaxial configuration enables movement of the plate 50 with respect to the screw 30 along a plurality of axes. In the configuration 70, the upper thread 34 and the upper threads 36 of the screw 30 are also engaged to the partial threads 58 of the plate 50 causing an effective lock between the screw 30 and the plate 50.

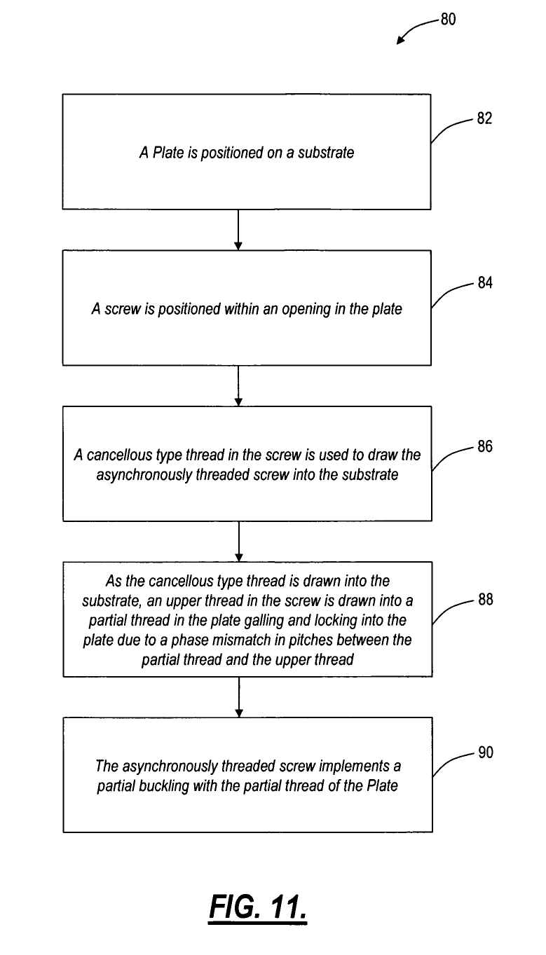

FIG. 11 is a flowchart of a plate retaining mechanism 80 for retaining a plate to a substrate with an asynchronous thread form according to an exemplary embodiment of the present invention. A plate is positioned on a substrate (step 862). A screw is positioned within an opening in the plate (step 84). A cancellous-type thread in the screw is used to draw the asynchronously threaded screw into the substrate (step 86). As the cancellous-type thread is drawn into the substrate, an upper thread in the screw is drawn into a partial thread in the plate galling and locking into the plate due to a phase mismatch in pitches between the partial thread and the upper thread (step 88). The asynchronously threaded screw implements a partial buckling with the partial thread of the plate (step 90).

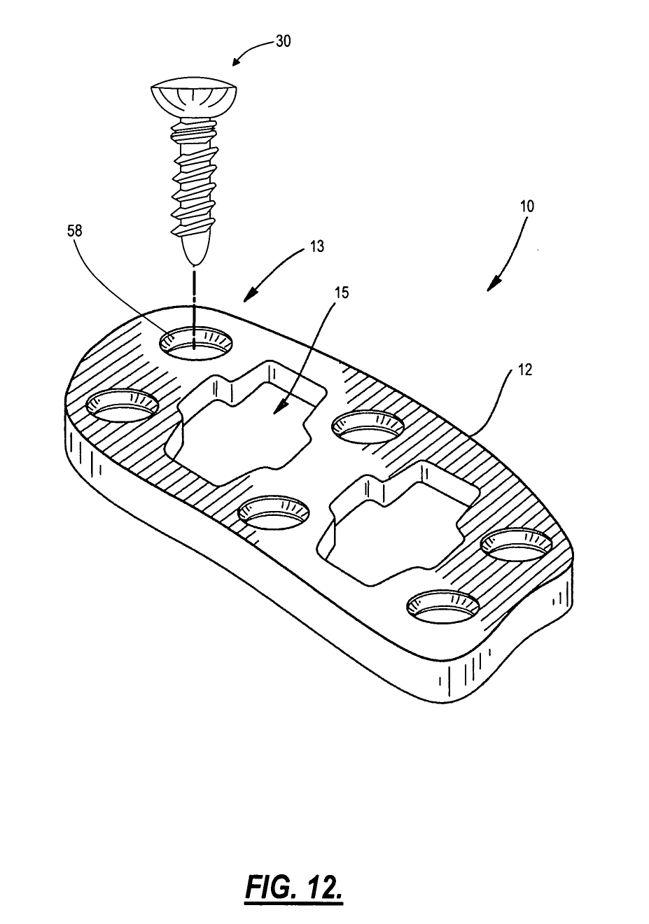

FIG. 12 is an exploded perspective view of a cervical plate 12 with a plurality of recessed screw holes 13 disposed there through for receiving a locking screw 30 according to an exemplary embodiment of the present invention. Each of the recessed screw holes 13 can include the partial threads 58 and side walls 54, 56 for receiving the screw 30. The cervical plate 12 and the screws 30 can be utilized to spinal procedures for effectively securing the cervical plate to the spine using the screws 30.

The locking screws 30 are disposed through the recessed screw holes 13 and driven into the bony vertebral structure beneath the cervical plate 12, thereby securing the cervical plate 12 to the bony vertebral structure and galling and locking to the cervical plate 12 through the mechanisms described herein. Preferably, the cervical plate screws 30 are positioned to correspond with adjacent vertebral levels of the cervical spine.

The cervical plate 12 is manufactured from a surgically implantable biocompatible material, such as a selected metal or composite, and has a length/width on the order of a few centimeters and a thickness on the order of a few millimeters. The cervical plate screws 30 are also manufactured from a surgically implantable biocompatible material, such as a selected metal or composite, and have a length on the order of a few millimeters to a few centimeters. It will be readily apparent to those of ordinary skill in the art that the cervical plate screws 30 can be manufactured to accept a hex driver or any other suitable driver, and can be replaced with other securing/actuating mechanisms that would perform similar functions. Preferably, once in place, the heads 32 of each of the cervical plate screws 30 sits substantially flush with the surface of the cervical plate 12.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A system for retaining a plate to a substrate with an asynchronous thread form, comprising:
a plate defining one or more holes each configured to receive a locking screw, wherein the plate comprises an outer surface, an interior portion, and an inner surface, each of the one or more holes having an inside diameter that is smaller adjacent to the outer surface than at the interior portion and partial threads disposed in the inner surface; and
one or more locking screws, each comprising:
a first threaded portion configured to pass through one of the one or more holes of the plate and securely engage a substrate; and
a second threaded portion configured to pass through one of the one or more holes of the plate and securely engage the partial threads, wherein the first threaded portion is asynchronous from the second threaded portion.

2. The system of claim 1, the one or more locking screws each further comprising:
a head portion attached to the second threaded portion configured to securely engage a lip structure associated with one of the one or more holes of the plate.

3. The system of claim 1, wherein the second threaded portion comprises threads out-of-phase with the partial threads disposed in the inner surface.

4. The system of claim 3, wherein the second threaded portion is configured to gall and lock causing partial buckling with the partial threads responsive to an applied torque thereby preventing reverse threading or backing out of the locking screw from the plate.

5. The system of claim 1, wherein the second threaded portion comprises threads with one or more different values for pitch, major diameter, minor diameter, root to crest distance, and thread angle from the partial threads disposed in the inner surface.

6. The system of claim 1, wherein the second threaded portion comprises threads with one or more different values for pitch, major diameter, minor diameter, root to crest distance, and thread angle from the first threaded portion.

7. The system of claim 1, wherein the first threaded portion comprises a cancellous type thread operable to draw the screw into the substrate.

8. The system of claim 1, wherein the substrate comprises a bone, and wherein the plate comprises a cervical plate.

9. The system of claim 1, wherein each of the one or more holes defined by the plate is configured to receive one of the one or more locking screws one of perpendicularly and at an angle.

10. A locking screw operable to retain a plate to a substrate, comprising:
an elongated member comprising:
an end portion operable to engage a substrate;
a middle portion; and
an upper portion;
first threads on the middle portion of the elongated member;
second threads on the upper portion of the elongated member, wherein the first threads and the second threads are asynchronous; and
a head disposed to the elongated member.

11. The locking screw of claim 10, wherein the head comprises a notch configured to securely engage a lip structure associated with a hole in the plate.

12. The locking screw of claim 10, wherein the second threads are out-of-phase with partial threads disposed in an inner surface of a hole in the plate.

13. The locking screw of claim 13, wherein the second threads are configured to gall and lock causing partial buckling with the partial threads responsive to an applied torque thereby preventing reverse threading or backing out of the locking screw from the plate.

14. The locking screw of claim 10, wherein the second threads comprise one or more different values for pitch, major diameter, minor diameter, root to crest distance, and thread angle from partial threads disposed in an inner surface of a hole in the plate.

15. The locking screw of claim 10, wherein the second threads comprise one or more different values for pitch, major diameter, minor diameter, root to crest distance, and thread angle from the first threads.

16. The system of claim 10, wherein the first threads comprise cancellous type threads operable to draw the screw into the substrate, wherein the substrate comprises a bone, and wherein the plate comprises a cervical plate.

17. A method for retaining a plate to a substrate with an asynchronous thread form, comprising:
positioning plate defining one or more holes on a substrate, wherein the one or more holes each configured to receive a locking screw, wherein the plate comprises an outer surface, an interior portion, and an inner surface, each of the one or more holes having an inside diameter that is smaller adjacent to the outer surface than at the interior portion and partial threads disposed in the inner surface;
positioning a locking screw within one of the one or more holes in the plate;
applying a torque to a head portion of the locking screw to draw the locking screw into the substrate through a first threaded portion of the locking screw; and
galling and locking a second threaded portion of the locking screw to a partial thread in the one of the one or more holes in the plate as the locking screw is drawn into the substrate.

18. The method of claim 17, further comprising:
implementing a partial buckling between the second threaded portion of the locking screw and the partial threads of the plate based on asynchronous threading of the second threaded portion in relation to the partial threads.

19. The method of claim 18, wherein the second threaded portion comprises threads with one or more different values for pitch, major diameter, minor diameter, root to crest distance, and thread angle from the partial threads.

20. The method of claim 18, wherein the second threaded portion comprises threads with one or more different values for pitch, major diameter, minor diameter, root to crest distance, and thread angle from the first threaded portion.
